(19) United States
(12) Patent Application Publication
(10) Pub. No.: US 2005/0255459 A1
(43) Pub. Date: Nov. 17, 2005

(54) Process and apparatus for using the sets of pseudo random subsequences present in genomes for identification of species

(76) Inventors: Yuriy Fofanov, Woodlands, TX (US); Bernard M. Pettitt, Houston, TX (US); Tongbin Li, Houston, TX (US); Serguei Tchoumakov, The Woodlands, TX (US)

Correspondence Address: RICHARD COALE WILLSON JR, 3205 HARVEST MOON DR, STE 200, PALM HARBOR, FL, 34683-2127, US

(21) Appl. No.: 10/879,061

(22) Filed: Jun. 30, 2004

Related U.S. Application Data
(60) Provisional application No. 60/483,682, filed on Jun. 30, 2003.

(51) Int. Cl. C12Q 1/70; C12Q 1/68; G06F 19/00; G01N 33/48; G01N 33/50

(52) U.S. Cl. 435/5; 435/6; 702/20

(57) ABSTRACT

Our research conducted with the genome sequences of more than 250 species of organisms (including viral, microbial, and multi-cellular organisms, and human) results in the discovery that the occurrence of a particular subsequence (the so-called “motifs” or “n-mers,” (n being the length of the subsequences), which can be up to 25 and higher) in the genome of a particular species can be considered as a nearly random event; and that the occurrences of a particular subsequence in the genome sequences of different species can be considered as nearly independent events (with the exception of the cases where extremely closely related species are compared). The set of subsequences that occur in a particular species' genome can therefore be used as a genomic “fingerprint” of this species. This discovery leads to the concept of utilizing a set of pseudo-randomly designed subsequences for species identification or discrimination. These subsequences (probes, primers, motifs, n-mers) can be used with hybridization-based technologies (including, but not limited to, the microarray or PCR technologies) and any other technology allow to identity the fact of presence/absence of particular subsequence in genomic DNA for identification of species. The same approach can also be used to identify individuals of the same species (including the human species), to estimate the genome size of unknown organisms, and to estimate the total genome size in samples containing several viral, microbial, and eukaryotic genomes. The identification methods currently in use for these purposes require sequencing of the genomic sequences of the species or the individuals of interest. The introduction of the proposed computational method eradicates such requirement, and will tremendously reduce the expense of these tests.

The present application claims priority of provisional U.S. Ser. No. 60/483,682 filed 30 Jun. 2003 (Attorney Docket 016APR/UH2317) by the same inventors, the entire contents of which is hereby incorporated by reference into this application.

### STATEMENT REGARDING FEDERALLY SPONSORED RESEARCH OR DEVELOPMENT

This invention was made with government support under Cooperative Agreement awarded by The National Institute of Health. The government possibly has certain rights in the invention,

### BACKGROUND OF THE INVENTION

1. Field of the Invention

This invention relates to the discipline of bioinformatics to the identification of species (viruses, microbes, multicellular organisms including human) or individuals using information about presence/absence of short subsequences (also called n-mers, where n stands for the length of the subsequence or motifs) in they genomes. Specifically this invention prefers use of subsequence of size 7≦n≦25.

2. Background of the Art

Over the past decade, the sequences of a large number of genomes (including viral, microbial, eukaryotic organisms [including that of human]) have become available in the public domain (see for example: http://www.ncbi.nlm.nih.gov/). The sequencing of more genomes is currently underway. This invention applies to the area of identifying species (including viral, microbial, and lower eukaryotic pathogens) and individuals (including, but not limited to, individual human beings) based on the differences in their genome sequences.

In the last several years, the use of combinatorial detection and synthesis technologies has qualitatively changed many areas of bioscience. These technologies include DNA, arrays, peptide arrays, protein arrays, combinatorial chemistry arrays and parallel PCR technologies. These technologies allow simultaneous parallel measurement of thousands of interactions on a biological sample.

This invention is based partially on statistical analysis of the occurrences of short subsequences in the genomes of about 250 species. However, the result of our analysis extends beyond these species. In fact, this invention covers the identification of any species, and any individuals based on the occurrences of short subsequences in their genomes.

Before the work leading to this invention, several attempts (Deschavanne et al. 1999; Karlin and Ladunga 1994; Karlin et al. 1997; Nakashima et al. 1997; Nakashima et al. 1998; Nussinov 1984; Sandberg et al. 2001) have been made to employ the frequency distributions of n-mers to analyze species with relatively short genome sizes (microbial). In such an approach, the shape of the frequency distribution for particular short subsequences (2-4-mers (Campbell et al. 1999; Karlin and Ladunga 1994, Karlin et al. 1997; Nakashima et al. 1997; Nakashima et al. 1998; Nussinov 1984) and 8-9-mers (Deschavanne et al. 1999; Sandberg et al. 2001) have been proposed as a measure to decide what microbial genome we are dealing with, based on a given random piece of genome or a whole genome. Included in this application is a consideration and description of the similarity of n-mers in various species and the deviation of the distribution of their presence from the random (Poisson) distribution

### SUMMARY OF THE INVENTION

The present invention details the results of a correlation analysis for distributions of the presence/absence of short subsequences of different length (n-mers, preferably 5≦n≦20) in more than 250 microbial and viral genomes and five genomes of multicellular organisms (including human). The results show that for organisms that are not close relatives of each other, a range of values of n can be found, such that the presence/absence of different n-mers in different genomes are practically not correlated (within a probabilistic tolerance, ε). For close relatives such correlations appear, but are not as strong as might be expected.

The absence of correlation among the n-mers present in different genomes leads to the possibility of using random sets of n-mers (with appropriately chosen n) to discriminate between different microbial and viral genomes and individual organisms including human beings. The discrimination is based on uniqueness of the combination of presence/absence of n-mers in each individual genome. The formulas derived yield the size of a experiment designed to identify an organism given the length of its genome, a convenient length of probe, n, and a tolerance or error, ε.

No such study has been found in the literature for n>11, due to the rapid increase of the computational complexity associated with previous algorithms. To be able to perform these calculations for these values n, new algorithms and specific data structures have been developed and implemented. The important advantage of this invention's approach is that it can be used without a priori knowledge of the sequence itself and the presence/absence of short n-mers in genomes can be counted in a reasonable amount of computing time.

The implication is there is no need to perform the expensive and time-consuming process of sequencing before array construction. Taking into account how accessible the DNA of thousands of viruses, microbes, and multicellular organisms is, how easily each analysis of the presence/absence of n-mers in any genome can he accomplished by using such techniques as PCR, oligonucleotide microarrays, etc., and the fact that one do not need to determine quantitative values of appearance (we need just a yes/no answer)—it is possible to produce essentially universal species identification devices.

### BRIEF DESCRIPTION OF THE DRAWINGS

For a detailed understanding and better appreciation of the present invention, reference should be made to the following detailed description of the invention and the preferred embodiments, taken in conjunction with the accompanying drawings.

FIGS. 1-3 show schematically a preferred embodiment of the apparatus.

FIG. 4 The frequency of presence of different n-mers, p=N(n, G)/4ⁿ, as a function of the ratio 4ⁿ/M for 70+ microbial genomes.

FIGS. 5-7 correspond to the microbial, RNA-containing viruses and DNA-containing viruses, respectively. The frequency of n-mers for different values of n is shown with different symbols. The analytical prediction that corresponds to the frequency of presence of n-mers in a purely random “genome” is also shown for comparison in all Figures as a solid line. One can observe the extraordinary similarity between these plots. All of the different genomes form a well-defined pattern, when plotted against the ratio 4ⁿ/M and not against the size of the genome or the length of the n-mer separately.

FIG. 6. Frequency of presence of 7-10-mers in 129 RNA viral genomes.

FIG. 7. Frequency of presence of 7-10-mers in 48 DNA viral genomes

FIG. 8. shows Frequency of presence of 7-10-mers in 48 DNA viral genomes

Supplemental Table 1S shows Frequency of presence of 8-mers and self-similarity for several viral genomes.

Supplemental Table 2S. Frequency of presence of 12-mers and self-similarity for several microbial genome

Table 1. The frequency of presence of 12-mers within the 3 microbial genome.

Table 2. Actual and predicted simultaneous presence of 12-mers within the 3 microbial genomes: (1) Salmonella typhi, (2) Mycobacterium tuberculosis H37Rv, and (3) Bacillus subtilis.

Table 3 The optimal length of n-mers (n*) for different genome sizes and frequencies of presence (p*).

Table 4. shows Actual and predicted simultaneous presence of 12-mers within the 3 extremely close microbial genomes: (a) Chlamydophila pneumoniae CWL029, (b) Chlamydophila pneumoniae AR39, and (c) Chlamydophila pneumoniae J138.

Table A provides Preferred, More Preferred, and Most Preferred levels for parameters of the invention.

### Additional Figures

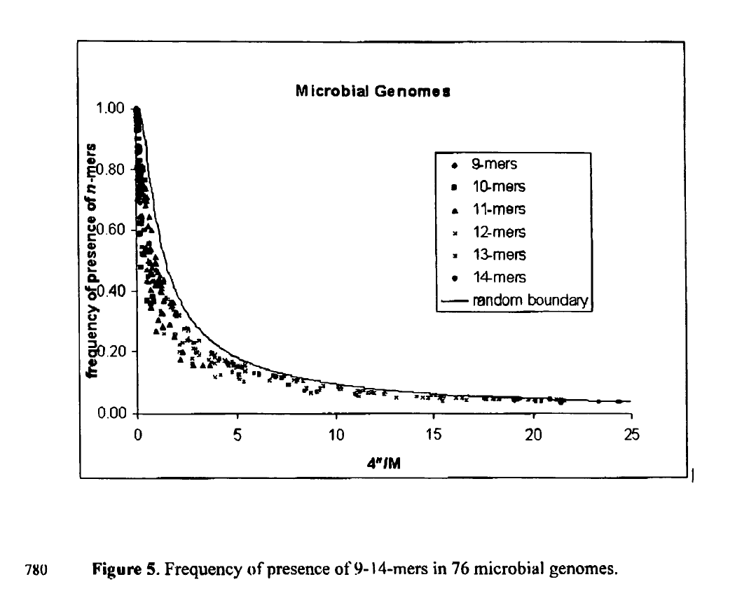

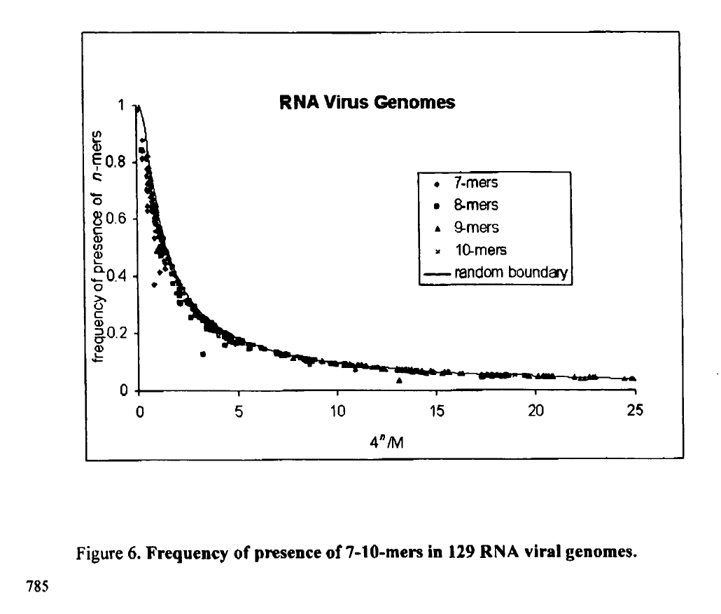

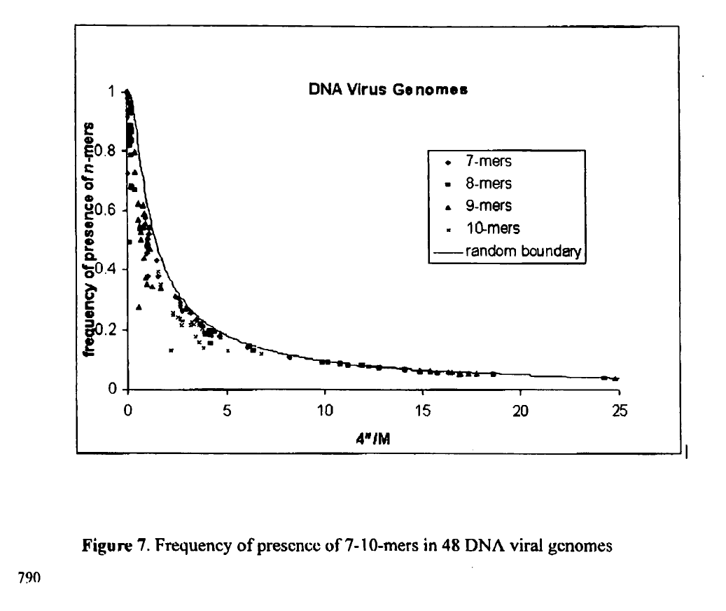

FIGS. 5-7 correspond to the microbial, RNA containing viruses and DNA containing viruses, respectively. The frequency of n-mers for different values of n is shown with different symbols. The analytical prediction that corresponds to the frequency of presence of n-mers in a purely random “genome” is also shown for comparison in all Figures as a solid line. One can observe the extraordinary similarity between these plots. All of the different genomes form a well-defined pattern, when plotted against the ratio 4ⁿ/M and not against the size of the genome or the length of the n-mer separately.

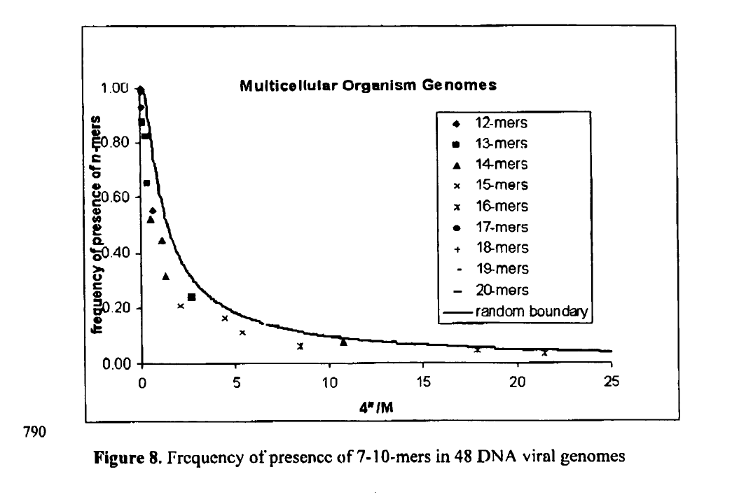

For much longer genomes of multicellular organisms practically all n-mers for n<12 are present. Therefore, we chose to calculate the number of distinct 13-20-mers present in each genome (see FIG. 8 and corresponding table below). These results point to the conclusion that the presence of namers in all genomes considered (in the range of n, when the condition M<<4ⁿ holds, where M is the genome length) can be treated as a nearly random process.

| | | | | Random | |
|---|---|---|---|---|---|
| | Total | Number of | Percent of | boundary: | |
| | Sequence | present n- | present n- | (1 − exp(−1/ | Self- |
| Genome | length (bp) | mers | mers | x)) | similarity |
| Caenorhabditis | 199,980,344 | 83,915,577 | 31.26% | 52.53% | 40.5% |
| elegans (14-mers) | | | | | |
| Drosophila | 239,963,692 | 119,253,045 | 44.43% | 59.10% | 24.8% |
| melanogaster (14- | | | | | |
| mers) | | | | | |
| Oryza sativa (15- | 511,742,384 | 220,383,196 | 20.52% | 37.91% | 45.9% |
| mers) | | | | | |
| Schizosaccharomyces | 24,980,160 | 9,256,101 | 55.17% | 31.08% | 28.8% |
| pombe (12-mers) | | | | | |
| Homo Sipiens 16- | 5,749,472,188 | 1,577,086,225 | 36.72% | 73.78% | 50.2% |
| mers | | | | | |

Frequency of presence of n-mers and self-similarity for several genomes of multicellular organisms (n is different for every genome).

### Supplemental Tables

Tables 1 and 2 show representative results for some of the analyzed genomes (microbial and viral), for n=8 and 12. It is worth mentioning that as n increases, the total number of possible n-mers, 4ⁿ, strongly exceeds the total sequence length M and most of the possible n-mers do not appear at all because the maximum number of n-mers contained in this sequence is M−n+1≈M. Moreover, for a reasonably high ratio, 4ⁿ/M, most of the n-mers which appear tend to appear only once, in accordance with the fact that the number of present n-mers becomes very close to M (see Tables 1,2 and supplementary data). That is why it was decided to use the statistics for “presence/absence” in our method of analysis, instead of the usual “frequency of appearance”, which is reasonable for short n-mers (total sequence length M<<4ⁿ).

SUPPLEMENTAL TABLE 1
Frequency of presence of 8-mers and self-similarity
for several viral genomes.

| | | Total | Number | Frequency | | |
|---|---|---|---|---|---|---|
| | | Sequence | of | of | | |
| | | length | presence | presence | Random | Self- |
| Accession | Genome | (bp) | 8-mers | 8-mers | boundary | similarity |
| NC_001436 | Human T-cell | 17,014 | 13,739 | 20.96% | 22.86% | 8.31% |
| | lymphotropic virus | | | | | |
| | type 1 | | | | | |
| NC_001707 | Hepatitis B virus | 6,430 | 5,963 | 9.10% | 9.35% | 2.64% |
| NC_001503 | Mouse mammary | 17,610 | 14,307 | 21.83% | 23.56% | 7.35% |
| | tumor virus | | | | | |
| NC_001547 | Sindbis Virus | 11,703 | 10,431 | 15.92% | 16.35% | 2.67% |
| NC_001434 | Hepatitis E virus | 7,176 | 6,517 | 9.94% | 10.37% | 4.12% |
| NC_003312 | Swine hepatitis E | 7,257 | 6,608 | 10.08% | 10.48% | 3.81% |
| | virus | | | | | |
| NC_001489 | Hepatitis A virus | 7,478 | 6,543 | 9.98% | 10.78% | 7.42% |
| NC_001433 | Hepatitis C virus | 9,413 | 8,480 | 12.94% | 13.38% | 3.29% |
| NC_001653 | Hepatitis D virus | 1,682 | 1,608 | 2.45% | 2.53% | 3.17% |
| NC_001802 | Human | 9,181 | 7,725 | 11.79% | 13.07% | 9.83% |
| | immunodeficiency | | | | | |
| | virus type 1 | | | | | |
| NC_003461 | Human | 15,600 | 12,242 | 18.68% | 21.18% | 11.82% |
| | parainfluenza virus 1 | | | | | |
| NC_001796 | Human | 15,462 | 11,506 | 17.56% | 21.02% | 16.46% |
| | parainfluenza virus 3 | | | | | |
| NC_003443 | Human | 15,646 | 12,702 | 19.38% | 21.24% | 8.74% |
| | parainfluenza virus 2 | | | | | |

SUPPLEMENTAL TABLE 2
Frequency of presence of 12-mers and self-similarity
for several microbial genomes.

| | | Total | | Frequency | | |
|---|---|---|---|---|---|---|
| | | Sequence | Number | of | | |
| | | length | of present | present | Random | Self- |
| Accession | Genome | (bp) | 12-mers | 12-mers | boundary | similarity |
| NC_000964 | Bacillus subtilis | 8,429,628 | 5,346,103 | 31.87% | 39.50% | 19.32% |
| NC_002696 | Caulobacter crescentus | 8,033,894 | 3,399,234 | 20.26% | 38.05% | 46.75% |
| NC_000913 | Escherichia coli K12 | 9,278,442 | 5,695,881 | 33.95% | 42.48% | 20.08% |
| NC_000916 | Methanobacterium | 3,502,754 | 2,658,450 | 15.85% | 18.84% | 15.91% |
| | thermoautotrophicum | | | | | |
| NC_003197 | Salmonella typhimurium | 9,714,864 | 5,821,910 | 34.70% | 43.96% | 21.06% |
| | LT2 | | | | | |
| NC_002758 | Staphylococcus aureus | 5,756,080 | 3,398,622 | 20.26% | 29.04% | 30.25% |
| | Mu50 | | | | | |
| NC_003098 | Streptococcus | 4,077,230 | 2,992,091 | 17.83% | 21.57% | 17.34% |
| | pneumoniae R6 | | | | | |
| NC_002737 | Streptococcus pyogenes | 3,704,882 | 2,778,223 | 16.56% | 19.81% | 16.43% |
| NC_002578 | Thermoplasma | 3,129,812 | 2,602,761 | 15.51% | 17.02% | 8.84% |
| | acidophilum | | | | | |
| NC_002689 | Thermoplasma | 3,169,608 | 2,590,718 | 15.44% | 17.22% | 10.30% |
| | volcanium | | | | | |
| NC_000919 | Treponema pallidum | 2,275,888 | 1,978,453 | 11.79% | 12.69% | 7.04% |
| NC_000853 | Thermotoga maritima | 3,721,450 | 2,755,886 | 16.43% | 19.89% | 17.43% |
| NC_002162 | Ureaplasma urealyticum | 1,503,438 | 948,274 | 5.65% | 8.57% | 34.06% |
| NC_002505 | Vibrio cholerae | 8,066,854 | 5,383,520 | 32.09% | 38.17% | 15.94% |
| | chromosome I, | | | | | |
| | chromosome II | | | | | |
| NC_002488 | Xylella fastidiosa 9a5c | 5,358,610 | 3,996,398 | 23.82% | 27.34% | 12.88% |

### DETAILED DESCRIPTION OF THE INVENTION

The use of novel detection and synthesis technologies has qualitatively changed many areas of bioscience in the last several years. These technologies include DNA, arrays, peptide arrays, protein arrays, combinatorial chemistry arrays and parallel PCR technologies. These technologies allow simultaneous, parallel measurement of thousands of interactions on a biological sample.

Over the past decade, the sequences of a large number of genomes (including viral, microbial, eukaryotic organisms |including that of human]) have become available in public domain (see for example http://www.ncbi.nlm.nih.gov/). The sequencing of more genomes is currently underway. This invention applies in the area of identifying species (including viral, microbial, and lower eukaryotic pathogens) and individuals (including, but not limited to, individual human beings) based on the differences in their genome sequences. In particular on the information regarding the presence/absence in the genome randomly or substantially randomly (e.g. filtered using particular criteria such as GC content, melting temperature, presence/absence in another genome, etc.) chosen short subsequences of size preferably up to 25 nucleotides.

This invention is based partially on the statistical analysis of the occurrences of short subsequences in the genomes of about 250 species. However, the result of the analysis extends beyond these species. In fact, this invention covers the identification of any species, and any individuals based on the occurrences of short subsequences in their genomes.

Before the work leading to this invention, several attempts (Deschavanne et al. 1999; Karlin and Ladunga 1994; Karlin et al. 1997; Nakashima et al. 1997; Nakashima et al. 1998; Nussinov 1984; Sandberg et al. 2001 ) have been made to employ the frequency distributions of n-mers to analyze species with relatively short genome sizes (microbial). In such an approach, the shape of the frequency distribution for particular short subsequences (2-4-mers (Campbell et al. 1999; Karlin and Ladunga 1994; Karlin et al. 1997; Nakashima et al. 1997; Nakashima et al. 1998; Nussinov 1984) and 8-9-mers (Deschavanne et al. 1999; Sandberg et al. 2001) have been proposed as a measure to decide what microbial genome we are dealing with, based on a given random piece of genome or a whole genome. Included in the invention below is a consideration and description of the similarity of n-mers in various species and the deviation of the distribution of their presence from the random (Poisson) distribution.

The principal goal of the research for this invention was to find how independent/correlated the appearances of n-mers arc in different genomes. The present invention approaches this question by using the well-known multiplication property for the joint probability of the intersection of events, according to which two events A, and B can be treated as independent if
p(A∩B)=p(A)p(B).

A simple example is based on 3 different genomes: (1) Salmonella typhi (NC_{—}003198), (2) Mycobacterium tuberculosis H37Rv (NC_{—}000962), and (3) Bacillus subtilis (NC_{—}000964). A complete set of n-mers would contain 4ⁿ n-mers, which, for n=12, is 4¹²=16,777,216, Using complete genome sequences we can calculate how many different 12-mers are contained in each of these three genomes (Table 1).

TABLE 1
The frequency of presence of 12-mers within
the 3 microbial genomes.

| | | | Number of | |
|---|---|---|---|---|
| | | | different 12- | |
| | | | mers | |
| | | | present in | |
| | Genome | | genome: | p = |
| Genome | length | TSL (M) | N(12, G) | N(12, G)/4ⁿ |
| (1) Salmonella | 4,809,037 | 9,618,074 | 5,813,330 | 34.65% |
| typhi | | | | |
| (2) Mycobacterium | 4,411,529 | 8,823,058 | 4,361,508 | 26.00% |
| tuberculosis | | | | |
| H37Rv | | | | |
| (3) Bacillus | 4,214,814 | 8,429,628 | 5,346,103 | 31.87% |
| subtilis | | | | |

To estimate the probability of finding randomly picked 12-mers in each genome, the frequency of presence of 12-mers calculated in each genome. These values are also presented in Table 1. Note the modest percentage when compared with the maximum of possible sequences, 4ⁿ.

The number N (n, G₁, G₂) of n-mers (n=12) that appear in each pair of species has also been computed (Table 2). Based on this we can compare the probabilities of finding randomly picked 12-mers in each pair of genomes with probabilities calculated using the multiplication rule. As seen from Table 2, the actual and calculated (expected) probabilities do not differ greatly from each other, which allows us to treat the presence/absence of randomly picked 12-mers in these 3 genomes as independent events.

TABLE 2
Actual and predictcd simultaneous presence of 12-mers
within the 3 microbial genomes: (1) Salmonella typhi,
(2) Mycobacterium tuberculosis H37Rv, and (3) Bacillus subtilis.

| | | | Calculated |
|---|---|---|---|
| | | | probability |
| | Number 12- | | assuming |
| Case | mers | N(n, G1, G2)/4ⁿ | independence |
| Present in genomes | 1,943,814 | 11.6% | 9.0% |
| (1) and (2) | | | |
| Present in genomes | 2,335,710 | 13.9% | 11.0% |
| (1) and (3) | | | |
| Present in genomes | 1,334,288 | 8.0% | 8.3% |
| (2) and (3) | | | |

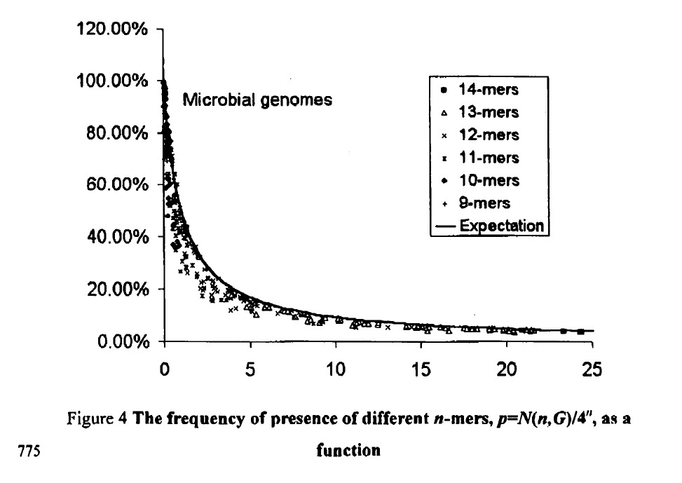

The actual and expected pair-wise probabilities were calculated in each above-mentioned group of genomes (170,000+ pairs in total). We were especially interested in the range of n where p*=5% -50% of the total possible number of n-mers occurred. This range is different for different genome sizes and can be determined from FIG. 4. The analytic formula for the random boundary also can be used to estimate this range:

$${n}^{*} = \frac{log [ M ( 1 - {p}^{*} ) / {p}^{*} ]}{log ( 4 )} . \qquad 2 )$$

Upper and lower bounds for sizes form 0.8 to 10 Mb, which are typical for microbial genomes, are shown in Table 3. In accordance with this, the value n=12 seems to be the most reasonable one for all microbial genomes. For viral genomes the value was found to be n=7.

TABLE 3
The optimal length of n-mers (n*) for different genome sizes and
frequencies of presence (p*).

| | Frequency | Frequency |
|---|---|---|
| | of presence | of presence |
| | 50% | 5% |
| TSL (M) | (p* = 0.5) | (p* = 0.05) |
| 0.8 Mb | 9.80 | 11.93 |
| 2.0 Mb | 10.47 | 12.59 |
| 10.0 Mb | 11.63 | 13.75 |

It was found that for all 2850 pairs of microbial genomes and the value of n=12 the average ratio of actual and expected probabilities is 1.35±0.61. For viral genomes and the corresponding value of n=7 the average ratio of actual and expected probabilities was found to be 1.06±0.10 for 1128 genome pairs DNA based viruses and 1.04±0.05 for 8128 genome pairs RNA based viruses. Thus, it is conclude that for this range of n the presences of n-mers in different genomes, to a good approximation, can be treated as independent events.

The highest deviations between *predicted and actual probabilities were found for closely related genomes. For 48 DNA-based viruses under consideration, using 7-mers, the highest ratio (185%) was found for Duck hepatitis B virus (NC_{—}001344) vs. Stork hepatitis B virus (NC_{—}003325) with 8.1% expected and 15.0% actual.

An example of closely related microbial genomes would be Staphylococcus aureus N315 (NC_{—}002745) vs. Staphylococcus aureus Mu50 (NC_{—}002758) with 4.0% *predicled and 19.7% actual or 491% higher than expected. Another extreme case was found for three microbial genomes: Chlamydophila pneumoniae CWL029(NC_{—}000922), Chlamydophila pneumoniae AR39 (NC_{—}002179), and Chlamydophila pneumoniae J138 (NC_{—}002491), which have the highest (8-fold) ratio of actual and expected probabilities for 12-mers (1.5%—expected and 12.3% actual). The results for these three microbial genomes are presented in Table 4.

TABLE 4
Actual and predicted simultaneous presence of 12-mers
within the 3 extremely close microbial genomes: (a)
Chlamydophila pneumoniae CWL029, (b)
Chlamydophila pneumoniae AR39, and (c) Chlamydophila
pneumoniae J138.

| | | | Calculated |
|---|---|---|---|
| | | | probability |
| | Number of | | assuming |
| Case | 12-mers | N(n, G₁, G₂)/4ⁿ | independence |
| Present in genome (a) | 7,712 | 0.046% | |
| and absent in genome | | | |
| (b) | | | |
| Absent in genome (a) | 7,214 | 0.043% | |
| and present in genome | | | |
| (b) | | | |
| Present in genomes | 2,058,304 | 12.268% | 1.52% |
| (a) and (b) | | | |
| Present in genome (a) | 11,526 | 0.069% | |
| and absent in genome | | | |
| (c) | | | |
| Absent in genome (a) | 10,706 | 0.064% | |
| and present in genome | | | |
| (c) | | | |
| Present in genomes | 2,054,490 | 12.246% | 1.52% |
| (a) and (c) | | | |
| Present in genome (b) | 6,939 | 0.041% | |
| and absent in genome | | | |
| (c) | | | |
| Absent in genome (b) | 6,617 | 0.039% | |
| and present in genome | | | |
| (c) | | | |
| Present in genomes | 2,058579 | 12.270% | 1.52% |
| (b) and (c) | | | |

For the group containing 24 human chromosomes pair-wise ratios of actual and expected probabilities of 14-mers were found to be 1.91±16, maximum ratio being found for n=20 and Y-chromosomes (expectation 2.9% vs. actual 6.9%).

Microbial/Viral Fingerprints Using Random Subsets of n-mers

Assuming that the results for 250+ genomes are statistically significant it is expected that similar behavior will be the case for many different (as yet sequenced) genomes. Thus the analysis indicates that, in this case, one may use relatively small sets of randomly picked n-mers for differentiating between different viruses and organisms.

The idea is illustrated by continuing our example for three microbial genomes. Let n* be the size of n-mer, which fits the interval where from 5% to 50% of all possible n-mers show up for a desirable rangc of genome lengths. In accordance with Table 3, the may the value n*=12 was chosen. Randomly picking L, 12-mers (say, L=1000). Given a genome G₁ with the frequency of presence of n-mers p₁, it is expected that K=p₁L n-mers present in G₁ will appear also in the random set, forming a “fingerprint” of G₁ (in the example, expect 50<K<500). The probability, ε, that the fingerprint of G₁ will exactly coincide with the fingerprint of some other genome G₂ (with the frequency of presence of n-mers p₂) is found in the Examples section. The result is
ε=(1−p₁−p₂+2p₁₂)^{L} 3)
Here p₁₂ is the probability for the n-mer to be present in both genomes simultaneously.

Considering the numeric example mentioned in Tables 1 and 2 of two species that are far from each other, Salmonella typhi vs. Mycobacterium tuberculosis H37Rv; p₁=0.3465, p₂=0.2600, p₁₂=0.1160; with L-1000 a remarkable accuracy of ε=1.7*10⁻²⁰⁴ can theoretically be achieved.

Given a desirable probability of error, ε, one can determine the appropriate size, L, of a random set of n-mers which can be used for reliable identification of genomes as

$$L = \frac{log ɛ}{log ( 1 - {p}_{1} - {p}_{2} + 2 {p}_{12} )} . \qquad 4 )$$

For related organisms, the genomes may contain large common parts. This means that p₁₂ may be close to p₁ and p₂. To give a numeric example of close relatives, consider Staphylococcus aureus N315 vs Staphylococcus aureus Mu50. Now p₁=0.198, p₂=0.203, p₁₂=0.197 and an accuracy of ε=10⁻¹⁰ can be achieved with L=4451. It is to be stressed the logarithmic dependence of the sampling or microarray size, L, on the error probability, ε. This feature is of principal importance for the estimation procedure under discussion.

Fingerprints of Closely Related Organisms

Next it is considered what happens when comparing closely related organisms using the above-described approach (e.g. different types of influenza or modifications of microbes). Assuming that two genomes G₁ and G₂ almost coincide and differ only in m randomly located characters (nucleotides). This situation simulates the existence of single nucleotide polymorphisms (SNPs). Let L be the size of the chip and p—the frequency of presence of n-mers in a genome with a TSL value M. The value of L, necessary to distinguish the fingerprints of these two genomes with the error probability ε, can be estimated by the formula (see Example 4):

$$L = \frac{log ɛ}{p log ( 1 - mn / N )} \leq \frac{M \vert log ɛ \vert}{pmn} . \qquad 5 )$$

Such an approach can provide the level of accuracy necessary for the individual human fingerprints. Assume that the differences between individual human beings appear only because of SNPs, which have equal probability and are randomly located in genome. According to literature estimates [13], the total number of SNPs in human genome is expected to be around 3,000,000. Then, calculating the necessary size for the random microarray (m/M˜0.1%, ε=10⁻¹⁰, n=17, p=0.284) we have L˜4769. This preliminary estimation is promising and indicates that this possibility deserves a proper experimental study. Recall that the theoretical estimations have been made for randomly-picked sets of n-mers. The further possibility exists to start with a larger than necessary random set of n-mers (say, L=10,000) and then to decrease the microarray size experimenting with the desirable set of genomes (using, for instance, an evolutionary optimization approach).

The analysis outlined in this invention predicts a logarithmic dependence of the sampling or microarray size, L, on the error probability, ε. This feature is of principal importance for the estimation procedure. Therefore, practically any sufficiently random subset of n-mers of appropriate size for design a microarray to diagnose to which organism a given DNA/RNA sample belongs may be employed. Different sizes of n-mers must be employed for recognition of different organisms based on their genome length. Values of n that correspond to given intervals of genome lengths can be easily calculated using the formulas outlined in this invention. Only 11 different n values, 7≦n≦17, would be sufficient to cover a large variety of genome sizes from 1 Kb to 9 Gb.

The important advantage the approach described in this invention is that it can be used without a priori knowledge of the sequence itself. The presence/absence of short n-mers in genomes can be counted in a reasonable amount of computing time when employing the newly designed algorithms and data structures devised and outlined in the invention above. This implies there is no need to perform the expensive and time-consuming process of sequencing before array construction. It is enough to obtain the purified DNA, hybridize it on a sufficiently random microarray chip and check which n-mers show up. Taking into account how accessible the DNA of thousands of microbial and viruses are, how easily each microarray can be produced, and the fact that we do not need to determine quantitative values or expression (we need just a yes/no answer)—it should be possible to produce an essentially universal microbial/viral DNA chip.

### EXAMPLES

The following examples are provided to illustrate the present invention. The examples are not intended to limit the scope of the present invention and they should not be so interpreted. Amounts, if any, are in weight parts or weight percentages unless otherwise indicated.

### Example 1

For our analysis we have picked genomes available in the NCBI [http://www.ncbi.nlm.nih.gov/entrez/query.fcgi?db=Genome] including microbial (76), viral (176), and multicellular organisms (5) genomes, with sizes ranging from 0.32 Kb (Cereal yellow dwarf virus-RPV satellite RNA NC_{—}003533) to 2.87 Gb (human). A complete list of all genomes and the complete results of the analysis discussed below are available as supplementary material at http://www.cs.uh.edu/˜bp/.

For our computations with multi-cellular organisms, microbial and viruses we used both complementary sequences for computational convenience because it is the way we can observe it based on the present technology (PCR, cDNA Microarrays, etc.). This trivially increases the amount of analyzed material by a factor of two. To take this fact into account for normalization, we will use the term “total sequence length”—TSL, equal to twice the genome. We will denote the total sequence length so defined by M.

As the first step of our analysis we have calculated the amount, N(n, G), of distinct 5-20 long n-mers present in each of 250+ considered genomes; here G stands for a genome. The corresponding results for 76 microbial genomes are shown in FIG. 4. The value of N(n, G) depends on two parameters: 4ⁿ—the total number of all possible n-mers, and the genome length, M. In FIG. 4 we show the frequency of presence of different n-mers, p=N(n, G)/4ⁿ, as a function of the ratio 4ⁿ/M. Note, that 4ⁿgrows very fast when n increases. For short n-mers, n<7, and long sequences, M>4ⁿ, a kind of “saturation” can be observed, when all or almost all possible n-mers are present in the sequence, In turn, when M<<4ⁿ, only a small part of the total number of n-mers appears, for instance in microbial genomes, where according to our observations most of them appear only once. The results for different M and n form a well-defined pattern. The upper bound of this pattern is given by a simple analytic formula, which can be found under assumption of the purely random appearance of n-mers in genomes:

$$p = \frac{1}{1 + \frac{{4}^{′′}}{M}} . \qquad 1 )$$

This statistical upper bound is shown in the figure as a solid line. Similar results for DNA and RNA based viruses and multi-cellular organisms can be found in supplementary data. It is worth noting that such a pattern for multi-cellular organisms is located notably below the expected upper bound, which can be explained by a significant presence of repeated parts in these genomes (Fofanov et al. 2002b).

Our second step was to study the presence/absence of short subsequences in more than one genome simultaneously. We performed such analyses separately in four different sets of genomes: RNA based viruses (128 genomes), DNA based viruses (48 genomes), Microorganisms (76 genomes) and Human. In each group the number of simultaneously present 5-18-mers were calculated for each pair of genomes. The fourth group contains 24 human chromosomes, for which the numbers of simultaneously present 7-20-mers were calculated for each pair of chromosomes.

### Example 2

Here we analytically estimate the frequency of presence of n-mers in a genome of length M. Let us apply the logic of the example shown in Tables 1 and 3 to autocorrelations, i.e. let us check whether the appearances of distinct n-mers are independent or correlated within a single genome. Assume that the multiple appearances of a given n-mer at different locations within the same genome are also independent events. Then, the probability of 12-mer to appear once is p, —twice=p², three times=p³ and so on. The total number of 12-mers in the genome, taking into account multiple appearances is
M≈4ⁿ(p+p²p³+ . . . )=4ⁿp/(1−p ), 6)
from which one obtains,
p≈M/(M+4ⁿ). 7)

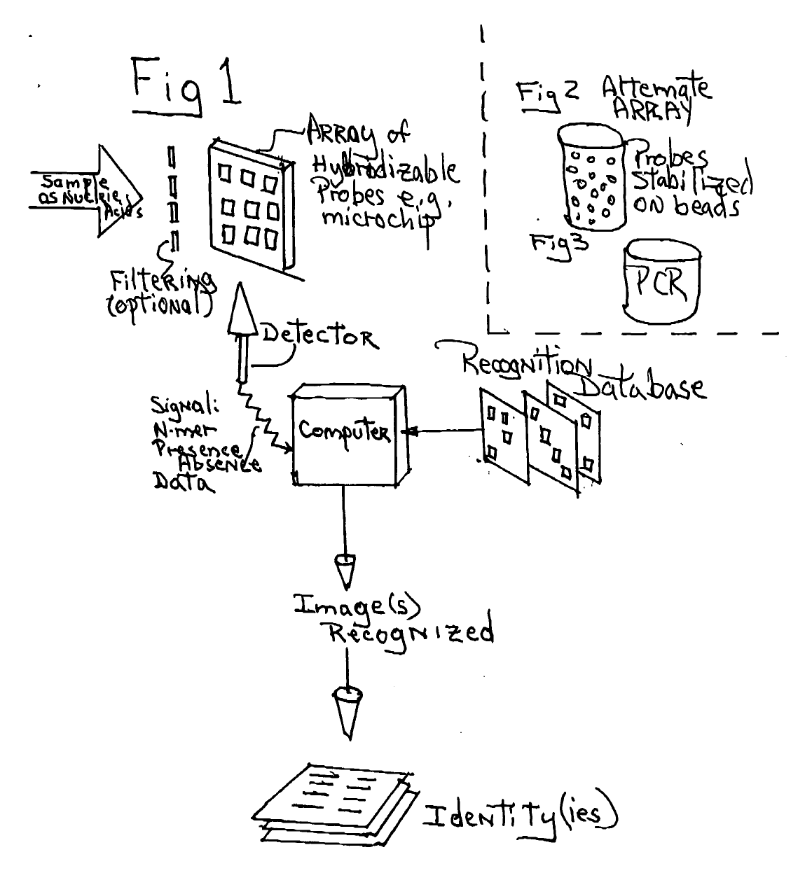

This formula has been presented in the text, and is shown in FIG. 1 by a solid line. One may also compare it to the experimental values from the last column of Table 1. In accordance with Eq. (1) we have for Salmonella typhi p=34.44%, for Mycobacteriiim tuberculosis H37Rv, p=34.46% and for Bacillius subtilis p=33.44%. This corresponds better to experimental values (34.65%, 26.00% and 31.87% respectively) than the estimation without taking into account multiple appearances,
p≈M/4ⁿ, 8)
which leads to the probabilities, 57.3%, 52.6% and 50.2% respectively. This fact is in accordance with the conclusion about the apparently nearly random statistical character of the appearance of n-mers in a single genome.

### Example 3

Here we will estimate the probability to make an error discriminating organisms by their analysis (“fingerprints”) in a random microarray, which consists of L n-mers. Assume that we need to discriminate between the two genomes G₁ and G₂ of sizes M₁ and M₂, respectively. Let G₁ (G₂) contains N₁ (N₂) different n-mers and N₁₂=N(n,G₁,G₂) n-mers are present simultaneously in both genomes (this is the size of intersection of two sets of n-mers corresponding to “n-mer contents” of G₁ and G₂; we denote this set as G₁∩G₂). The union G₁∪G² contains N₁+N₂−N₁₂ n-mers. Let us consider a fingerprint of the union of the two genomes, G₁∪G₂. For every n-mer appearing in this fingerprint, the probability that it occurs in the intersection region, G₁∩G₂, is

$$\frac{{N}_{12}}{{N}_{1} + {N}_{2} - {N}_{12}} . \qquad 9 )$$

An error, E, occurs when two genomes share the same fingerprint, i.e. all of n-mers that form the fingerprint represent the intersection region. This will happen with probability

$$P ( E \vert k ) = {( \frac{{N}_{12}}{{N}_{1} + {N}_{2 } - {N}_{12}} )}^{k} . \qquad 10 )$$

In fact, this is a conditional probability of an error, E, if we have a fingerprint of length k. We now need to calculate an average with respect to all possible fingerprints. There are

$${C}_{k}^{L} = \frac{L !}{k ! ( L - k ) !}$$

different fingerprints of the size k, which appear with equal probabilities [P(S ∈G₁∪G₂)]^{k}[1−P(S ∈G₁∪G₂)]^{L-k}, where P(S ∈G₁∪G₂) is the probability for n-mer S to find itself in the intersection G₁∪G₂ sampling L times. Therefore, we come to a binomial distribution of fingerprint sizes,

$$P ( k ) = {{\frac{L !}{k ! ( L - k ) !} [ \frac{{N}_{1} + {N}_{2} - {N}_{12}}{{4}^{′′}} ]}^{k} [ 1 - \frac{{N}_{1} + {N}_{2} - {N}_{12}}{{4}^{′′}} ]}^{L k} . \qquad 11 )$$

Calculating die average error we have,

$$P ( E ) = {\sum }_{k} P ( E \vert k ) P ( k ) = {( 1 - {p}_{1} - {p}_{2} + 2 {p}_{12} )}^{L} . \qquad 12 )$$

Here, pⱼ=Nⱼ/4ⁿ is the probability of presence in Gⱼ (j=1,2), and p₁₂ =n₁₂/4ⁿ is the probability of presence in the intersection G₁∩G₂. Given a desirable level of tolerance or error, P(E)˜ε, one can now estimate the appropriate combinatorial experiment (array) size:

$$L = \frac{log ɛ}{log ( 1 - {p}_{1} - {p}_{2} + 2 {p}_{12} )} . \qquad 13 )$$

We would like to again stress the logarithmic dependence of the microarray size L on the error level ε. This feature is of principal importance for the analysis under discussion. The following three cases will be considered separately.

### Example 4

Essentially different organisms. In this case, in accordance with the discussion in the text, the presence/absence of n-mers in one genome is not correlated with the presence/absence of n-mers in another genome and we can write p₁₂≈p₁p₂. Taking, for simplicity, p₁≈p₂≈p, we obtain,

$$L = \frac{log ɛ}{log ( 1 - 2 p + 2 {p}^{2} )} . \qquad 14 )$$

For instance, if ε=10⁻¹⁰ and p=0.05, we obtain L=230.

Related organisms. Now, p₁₂≠p₁p₂. Assuming that intersection G₁∩G₂ is almost coincides with the union, G₁∪G₂, or
N₁+n₁−N₁₂>N₁₂>>N₁+N₁−2N₁₂, 15)
one can rewrite Eqn. 12 in a slightly different form. Starting once again with Eqs. 10-12 and approximating the binomial distribution by the Gaussian of width s=\sqrt{LP(1−P)}, centered at k=LP where P=(N₁+N₂−N₁₂)/4ⁿ is the probability for an n-mer to be present in the union G₁∪G₂ we find,

$$P ( E ) = {\sum }_{k} {ⅇ}^{- \text{?}} \frac{1}{s \sqrt{2 π}} {ⅇ}^{- {( k - \overline{k} )}^{2} / 2 {s}^{2}} , \\ {ⅇ}^{- \text{?}} = \frac{{N}_{12}}{{N}_{1} + {N}_{2} - {N}_{12}} . \\ \text{?} \text{indicates text missing or illegible when filed} \qquad 16 )$$

Provided that α<<1 (which follows from inequality (5)) and \overline{k}>>1 (which is consistent with a small error level), one can change the summation to integration and obtain immediately,

$$P ( E ) = \frac{1}{s \sqrt{2 π}} \int {ⅇ}^{\text{?} {( k - \overline{k} )}^{2} / 2 {s}^{2}} ⅆ k = {ⅇ}^{α \overline{k} + {α}^{\text{?}} {s}^{\text{?}} / 2} . \\ \text{?} \text{indicates text missing or illegible when filed} \qquad 17 )$$

Finally,

$$P ( E ) \approx {( \frac{{N}_{12}}{{N}_{1} + {N}_{2} - {N}_{12}} )}^{\overline{k}} . \qquad 18 )$$

Now we can find the relation between the error level and the microarray size in the form,

$$\overline{k} = PL = \frac{log ɛ}{log [ {N}_{12} / ( {N}_{1} + {N}_{2} - {N}_{12} ) ]} . \qquad 19 )$$

Here, P, the probability of presence of n-mer in the intersection of two genomes, is given by P=(N₁+N₂−N₁₂)/4ⁿ˜p₁˜p₂. The last formula leads to similar numerical values as Eqn. (5) if N₁₂>>N₁+N₁−2N₁₂. Say, for P=0.05, N₁₂/(N₁+N₂−N₁₂)=0.9, ε=10⁻¹⁰, we have, L=4371.

Closely related organisms. Let us assume that two genomes G₁ and G₂ almost coincide and differ only in m randomly located characters (nucleotides). This situation simulates the existence of single nucleotide polymorphisms (SNPs). For simplicity, let us assume, that N₁=N₂=N. Every character that is different in G₁ and G₂ belongs simultaneously to n different n-mers, and the size of the subset in G₁∪G₂ a which consists of the n-mers that are different in G₁ and G₂ has a size, nm=2N−2N₁₂. Then,

$${N}_{12} = N - mn / 2 , or {N}_{1} + {N}_{2} - {N}_{12} = N + mn / 2 , \\ P ( E ) \approx {( 1 - \frac{nm}{N} )}^{\overline{k}} = ɛ . \qquad 20 )$$

Taking into account, that N≦M, we arrive at the estimation,

$$L = \frac{\overline{k}}{P} = \frac{log ɛ}{P log ( 1 - mn / N )} \leq \frac{M \vert log ɛ \vert}{Pmn} . \qquad 21 )$$

Table A gives preferred values for some of the parameters of the invention.

TABLE A

| Parameter | Preferred | More Preferred | Most Pref |
|---|---|---|---|
| Input Sample | Body Fluids (blood, urine, | Body fluids, | Body fluids, |
| | saliva, sputum, spcrm, biopsy | agricultural | PCR products |
| | sample, forensic samples, | products, | |
| | tumor cell, vascular placques, | microbial | |
| | transplant tiussues, skin, | colonies, PCR | |
| | urinefeces); Agricultural | products | |
| | Products (grains, seeds, plants, | | |
| | meat, livestock, vegetables, | | |
| | rumcn contents, etc.); soil, air | | |
| | particulates; PCR products; | | |
| | purified nucleic acids, | | |
| | amplified nucleic acids, | | |
| | natural waters, contaminated | | |
| | liquids; surface scrapings or | | |
| | swabbings; Animal RNA, cell | | |
| | cultures, pharmaceutical | | |
| | production cultures, CHO cell | | |
| | cultures, bacterial cultures, | | |
| | virus-infected cultures, | | |
| | microbial colonies | | |
| Target organisms | 10-1,000,000 | 2-20 | 1-2 |
| per sample | | | |
| Target sequence | GenomicDNA, Bacterial DNA | Virus RNA, Virus | genomic |
| type | Mitochondrial DNA, cDNA | DNA, genomic | DNA |
| | Virus DNA, virus RNA | DNA | |
| | PCR product, human DNA, | | |
| | human cDNA | | |
| Organism | Bacterium, virus, plant, | Bacterium, | Bacterium |
| | animal, fungus, yeast, mold, | Archaea, | |
| | Archae; Eukyarotes; Spore; | eukaryotic | |
| | Fish; Human; Gram-Negative | microorganism | |
| | bacterium, Y. pestis, HIV1, B.anthracis, | virus | |
| | Smallpox virus | | |
| Nucleic Acid | Chromosomal DNA; rRNA; | rRNA, Viral | chromosomal |
| | rDNA; cDNA; mt DNA, | RNA, Viral | DNA |
| | cpDNA, aRNA, plasmid | DNA, | |
| | DNA, oligonucleotides; PCR | chromosomal | |
| | product; Viral RNA; Viral | DNA | |
| | DNA; restriction fragment; | | |
| | YAC, BAC, cosmid | | |
| Probe length | 5 to 2500 | 7 to 20 | 10 to 20 |
| Number of probes | 1-100,000,000 | 20-100,000 | 50-10,000 |
| Classification | Kingdom; Phylum; Class; | Genus; Species, | Strain, |
| Level | Order; Family; Genus; | Strain | Species |
| | Species; Subgroups; Strain, | | |
| | Tribe, Scrotype; Gram stain | | |
| Utility | Clinical Diagnosis; Pathogen | Clinical | Clinical |
| | discovery; Biodefense; | Diagnosis; | Diagnosis |
| | Research; Adulterant | Biodefense; | |
| | Detection; Counterfeit | Adulterant | |
| | Detection; Food Safety; | Detection | |
| | Taxonomic Classification; | | |
| | Microbial ceology; | | |
| | Environmental Monitoring; | | |
| | Agronomy; Law Enforcement | | |
| Sample | acid, base, detergent, phenol, | Polymerase, | Polymerase, |
| preparation Agent | ethanol, isopropanol, | restriction | phenol |
| | chaotrope, enzyme, protease, | endonuclease, | |
| | nuclease, polymerase, | Phenol | |
| | adsorbent, ligase, primer, | | |
| | nucleotide, restriction | | |
| | endonuclease, detergent | | |
| Sample | Filter, Centrifuge, Extract, | Filter, centrifuge, | Fillter, culture |
| Preparation | Adsorb, protease, nuclease, | culture | |
| Pretreatment | partition, wash, leach, lyse, | | |
| | electrophoresis, precipitate, | | |
| | germinate, Culture | | |
| Hybridization | Aqueous buffer, solution | Aqueous buffer, | Solution |
| Medium | containing formamide, | solution | containing |
| | zwitterion solution, heated | containing | formamide, |
| | solution, alcohol solution | formamide, | heated |
| | | heated solution | solution |
| Cultivation Media | LB, M9, blood agar, DMEM, | LB, blood agar, | Blood agar |
| | calf serum medium, | Culture medium | |
| | McConkey's medium, Culture | containing host | |
| | medium containing host cells | cells | |
| Separation media | Ion exchanger, filter, | Ion exchanger, | Ion |
| for sample | ultrafilter, depth filter, | multiwell filter, | exchanger, |
| preparation | multiwell filter, centrifuge | immobilized- | silica, |
| | tube, multiwell plate, | metal affinity | magnetic |
| | immobilized-metal affinity | adsorbent, | beads |
| | adsorbent, hydroxyapatite, | multiwell plate, | |
| | silica, zirconia, magnetic | hydroxyapatite, | |
| | beads | silica, magnetic | |
| | | beads | |
| Detection Means: | Mass Spec.; Fluorescence; | Hybridization, | DNA probe |
| (Probe | Chemiluminesence; Enzyme | DNA probe array, | array |
| Hybridization): | Reaction; Radiochemical; | RT-PCR | |
| | Self-quenching Probe | | |
| | hybridization; Surface | | |
| | Plasmon Resonance; Total | | |
| | Internal Reflection | | |
| | Fluorescence; Liquid Crystals; | | |
| | Magnetic; Infrared; Array | | |
| | Detection Peptide Nucleic | | |
| | Acid hybridization; Branched | | |
| | DNA hybridization; Redox | | |
| | Chemistry; LNA | | |
| | hybridization, PNA | | |
| | hybridization, array, bead | | |
| | array | | |
| Detection Means: | Mass Spectrometry; | Mass | Mass |
| (Nonhybridization | Electrophoresis; Affinity | spectrometry, | spectrometry |
| Methods: | electrophoresis; | HPLC | |
| | Chromatography, IIPLC; | | |
| | DHPLC; Neutron Activation | | |
| | Analysis | | |
| Support | Array, chip, PCR, beads, etc. | | Microarray |

Modifications:

Specific compositions, methods, or embodiments discussed are intended to be only illustrative of the invention disclosed by this specification. Variations on these compositions, methods, or embodiments are readily apparent to a person of skill in the art based upon the teachings of this specification and are therefore intended to be included as part of the inventions disclosed herein.

Also it will be obvious to skilled persons that products and/or separation step techniques than other those recited herein may be used to great advantage in specific applications of the invention.

For example, the invention comprises:
A. A method for discriminating between organisms comprising different microbial-, viral- and individual human being-genomes, with a convenient number of combinatorial experiments by correlation analysis for distributions of the presence/absence of short subsequences of different length (n-mers) without requiring a priori knowledge of the sequence itself; said method comprising in combination the steps of:
   A. Obtaining a purified sample of DNA;
   B. Hybridizing the DNA onto a substantially combinatorial experimental platform;
   C. Determining which of certain n-mers are present in the hybridized DNA;
   D. Discriminating between different microbial and viral genomes based on the distribution of N-mers found.
B. The method of claim 1 wherein correlation analysis for distributions of the presence/absence of short subsequences of n-mers is used to discriminate between species.
C. The method of claim 1 wherein the number of combinatorial experiments to identify an organism is substantially chosen given the length of the genome of the organism, M; a convenient length of probe, n; and the tolerance or error, ε.
D. A method of identifying an organism, comprising in combination:
   a. Preparing nucleic acids from a sample containing the organism
   b. forming a presence/absence pattern by identifying the presence or absence of a plurality of specific subsequences in the nucleic acids
   c. comparing the determined presence/absence pattern with a computed pattern database to identify the organism preferably then identifying a set of organisms;and more preferably comparing this with a computed pattern.
E. A method of identifying viral, microbial and multi-cellular organisms based on the occurrence/absence of short subsequences in the genomes.
F. A method of identifying individuals of the same species, based on the occurrences of short subsequences in the genomes.
G. A method of identifying individual genome size of viral, microbial and multi-cellular organisms, based on the occurrences of short subsequences in the genomes.
H. An above method for identifying cumulative genome size of environmental or clinical samples or of samples containing mixed viral, microbial and multi-cellular organisms, based on the occurrences of short subsequences in the samples.
I. The method is developed based partially on the finding that the occurrences of short subsequences of size n, when n is properly chosen (for example when 4ⁿis bigger than length of genome(s) if of interest), is close to random; and that the occurrences of short subsequences between different species is close to independent.
J. The above methods wherein the set n-mers to be tested contain sequence of size from 7 to 25 nucleotides long and wherein the set n-mers to be tested is generated randomly and contains from 10 to 1000,000 sequences.
K. The above methods wherein the set n-mers to be tested is filtered or generated “quasi randomly” so all sequences have same or similar property selected from the group of properties consisting of: GC content, melting temperature (binding energy), presence or absence of same or similar pattern in certain position(s); can not hybridize to themselves or other sequences in the set); (for example particular nucleotide or combination of nucleotides).
L. The above methods wherein the set n-mers to be tested is generated “quasi randomly” so all sequences do not have particular pattern(s) (for example no sequences allow to have same nucleotide four or more times in lane).
M. The above methods wherein the set n-mers is tested by using any parallel detection techniques (including, but not limited to, DNA microarrays and parallel PCR, RT PCS, TaqMan, etc.).
N. A nucleic acid hybridization-based biosensing device comprising a) a support having at least one surface and b) a collection of probe molecules attached to the surface, each probe being unique and comprising a plurality of oligonucleotide probe molecules preferably having identical sequence within each distinct probe wherein the collection comprises a probe set. Preferably this is accomplished with 8-25 length probes, enough diversity in the probe set to generate useful patterns among an approx. infinite number of target populations, probes have predefined C-G base, C-G base variation forms a gradient, generate fingerprint hybridization pattern, etc.
O. Anc above method for identifying viral, microbial and multi-cellular organisms, and of identifying individuals of the same species, based on the occurrences of short subsequences in the genomes.
P. A method based partially on the finding that the occurrences of short subsequences of size n, when n is properly chosen, is close to random; and that the occurrences of short subsequences between different species is close to independent.
Q. A method in which randomly picked or quasi-randomly designed short oligomers are used in conjunction with parallel detection mechanisms (including, but not limited to, DNA microarrays and parallel PCR) to form a device to conveniently identity the organisms in a biological sample.
R. The method can be used to identify viral, microbial and multi-cellular pathogens contained in a biological sample. It can also be applied to identify the presence of any species, harmful or non-harmful, in any biological sample under other situations.
S. The method can also be used to identify an individual among other individuals within the same species based on the differences in the occurrences of short subsequences in their genomes. Applications include identifying an individual human being based on trace samples he/she leaves in a crime scene; and identifying/tracing individual livestock based on meat sample in the food supply that may have been inflicted by certain diseases (e.g., mad cow disease).
T. A method for identifying species or individuals within species comprising performing recognition analysis of present/absent patterns for selected n-mers, and comparing to such patterns for known moieties, to identity the biotechnical entity, without requiring prior knowledge of the genome sequences of the species or individuals to be identified.
U. A method of identifying individual genome size of viral, microbial and multi-cellular organisms, based on the occurrences of short subsequences in the genomes.
V. A method of identifying tile cumulative genome size of samples containing mix of many organisms (such as environmental or clinical samples), based on the occurrences of short subsequences in the samples under consideration.

Reference to documents made in the specification is intended to result in such patents or literature being expressly incorporated herein by reference.

### REFERENCES

Brenner, S., M. Johnson, J. Bridgham, G. Golda, D. H. Lloyd, D. Johnson, S. Luo, S. McCurdy, M. Foy, M. Ewan, R. Roth, D. George, S. Eletr, G. Albrecht, E. Vermaas, S. R. Williams, K. Moon, T. Burcham, M. Pallas, R. B. DuBridge, J. Kirchner, K. Fearon, J. Mao, and K. Corcoran. 2000. Gene expression analysis by massively parallel signature sequencing (MPSS) on microbead arrays. Nat Biotechnol 18: 630-634.
Campbell, A., J. Mrazek, and S. Karlin. 1999. Genome signature comparisons among prokaryoke, plasmid, and mitochondrial DNA. Proc Natl Acad Sci USA 96: 9184-9189.
Cutler, D. J., M. E. Zwick, M. M. Carrasquillo, C. T. Yohn, K. P. Tobin, C. Kashuk, D. J. Mathews, N. A. Shah, E. E. Eichler, J. A. Warrington, and A. Chakravarti. 2001. High-throughput variation detection and genotyping using microarrays. Genome Research 11: 1913-1925.
Deschavanne, P. J., A. Giron, J. Vilain, G. Fagot, and B. Fertil. 1999. Genomic signature: characterization and classification of species assessed by chaos game representation of sequences. Mol Biol Evol 16: 1391-1399.
Fislage, R. 1998. Differential display approach to quantitation of environmental stimuli on bacterial gene expression. Electrophoresis 19: 613-616.
Fislage, R., M. Berceanu, Y. Humboldt, M. Wendt, and H. Oberender. 1997. Primer design for a prokaryotic differential display RT-PCR. Nucleic Acids Res 25: 1830-1835.
Fofanov, Y., Y. Luo, C. Katili, J. Wang, B. Y., T. Powdrill, V. Fofanov, T.-B. Li, S. Chumakov, and B. M. Pettitt. 2002b. Short subsequences in genomes: How random are they? (submitted).
Forman, E. J., I. D. Walton, D. Stern, R. P. Rava, and M. O. Trulson. 1998. Thermodynamics of dupex formation and mismatch discrimination of photolithographically synthesized oligonucleotide arrays. ACS Symposium Series 682: 206-228.
Guo, Z., R. A. Guilfoyle, A. J. Thiel, R. Wang, and L. M. Smith. 1994. Direct flourescence analysis of genetic polymorphisms by hybridization with oligonucleotide arrays on glass supports. Nucleic Acids Res. 22: 5456-5465.
Heaton, R. J., A. W. Peterson, and R. M. Georgiadis. 2001. Electrostatic surface plasmon resonance: Direct electric field-induced hybridization and denaturation in monolayer nucleic acid films and label-free discrimination of base mismatches. Proceedings of the National Academy of Sciences of the United States of America 98, 3701-3704.
Karlin, S. and I. Ladunga. 1994. Comparisons of eukaryotic genomic sequences. Proc Natl Acad Sci U S A 91: 12832-12836.
Karlin, S. and J. Mrazek. 1997. Compositional differences within and between eukaryotic genomes. Proc Natl Acad Sci U S A 94: 10227-10232.
Nakashima, H., K. Nishikawa, and T. Ooi. 1997. Differences in dinucleotide frequencies of human, yeast, and Escherichia coli genes. DNA Res 4: 185-192.
Nakashima, H., M. Ota, K. Nishikawa, and T. Ooi. 1998.Genes from nine genomes are separated into their organisms in the dinucleotide composition space. DNA Res 5: 251-259.
Nguyen, T. T., A. Y. Grosberg, and F. I. Shklovskii. 2000. Screening of a charged particle by multivalent counterions in salty water: Strong charge inversion. J. Chem. Phys. 113: 1110-1125.
Nielsen, P. E. 2001. Peptide nucleic acid: a versatile tool in genetic diagnostics and molecular biology. Current Opinion Biotech. 12: 16-20.
Nussinov, R. 1984. Doublet frequencies in evolutionary distinct groups. Nucleic Acids Res 12: 1749-1763.
Peterson, A. W., R. J. Heaton, and R. M. Georgiadis. 2001. The effect of surface probe density on DNA hybridization. Nucleic Acids Res. 29: 5163-5168.
Sandberg, R., G. Winberg, C. I. Branden, A. Kaske, I. Ernberg, and J. Coster. 2001. Capturing whole-genome characteristics in short sequences using a naive Bayesian classifier. Genome Res 11: 1404-1409.
SantaLucia, J., H. T. Allawi, and P. A. Seneviratne. 1996. Improved nearest-neighbor parameters for predicting DNA duplex stability. Biochemistry 35: 3555-3562.
Shchepinov, M. S., S. C. Case-Green, and E. M. Southern. 1995. Steric factors influencing hybridization of nucleic acids to oligonucleotide. Nucleic Acids Res. 25: 1155-1161.
Southern, E. M. 2001. DNA microarrays—history and overview. Methods of Molecular Biology 170: 1-15.
Steel, A. B., T. M. Herne, and M. J. Tarlov. 1998. Electrochemical quantitation of DNA immobilized on gold. Anal. Chem. 70: 4670-4677.
Su, H. J., S. Surrey, S. E. McKenzie, P. Fortina, and D. J. Graves. 2002. Kinetics of heterogeneous hybridization on indium tin oxide surfaces with and without an applied potential. Electrophoresis 23: 1551-1557.
Vainrub, A. and B. M. Pettitt, Surface electrostatic effects in oligonucleotide microarrays: Control and optimization of binding thermodynamics. in press, Biopolymers.
Vainrub, A. and B. M. Pettitt. 2000. Thermodynamics of association to a molecule immobilized in an electric double layer. Chemical Physics Letters 323: 160-166.
Vainrub, A. and B. M. Pettitt. 2002. Coulomb blockage of hybridization in two-dimensional DNA arrays. Physical Review E 66: art. no.-041905.
Vasiliskov, V. A., D. V. Prokopenko, and A. D. Mirzabekov, 2001. Parallel multiplex thermodynamic analysis of coaxial base stacking in DNA duplexes by oligonucleotide microchips. Nucleic Acids Res. 29: 2303-2313.
Watterson, J. H., P. A. Piunno, C. C. Wust, and U. J. Krull. 2000. Effects of oligonucleotide immobilization density on selectivity of quantitative transduction of hybridization of inmmobilized DNA. Langmuir 16: 4984-4992.

1. A method for discriminating between different microbial-, viral- and individual human being-genomes, with a convenient number of combinatorial experiments by correlation analysis for distributions of the presence/absence of short subsequences of different length (n-mers) without requiring a priori knowledge of the sequence itself; said method comprising in combination the steps of
a. Preparing nucleic acids from a sample containing the organism;
b. Identifying the presence or absence of a plurality of subsequences in nucleic acids;
c. Comparing the presence/absence pattern with a database to discriminate between different microbial and viral genomes based on the distribution of N-mers found; preferably wherein the n-mers have length of 5-20.

2. The method of claim 1 wherein the n-mers have length of 5-20.

3. The method of claim 1 wherein correlation analysis for distributions of the presence/absence of short subsequences of n-mers is used to discriminate between species.

4. The method of claim 1 wherein the number of combinatorial experiments to identify an organism is substantially chosen given the length of the genome of the organism, M; a convenient length of probe, n; and the tolerance or error, ε.

5. The method of claim 1 wherein n is greater than 11.

6. A method of identifying an organism, comprising in combination:
a. Preparing nucleic acids from a sample containing the organism
b. forming a presence/absence pattern by identifying the presence or absence of a plurality of specific subsequences in the nucleic acids
c. comparing the determined presence/absence pattern with a database to identify the organism.

7. A method of claim 1 for identifying cumulative genome size of environmental or clinical samples or of samples containing mixed viral, microbial and multi-cellular organisms, based on the occurrences of short subsequences in the samples.

8. A method of claim 1 based partially on the finding that the occurrences of short subsequences of size n, when 4ⁿ is bigger than length of genome(s) of interest), is substantially random; and that the occurrences of short subsequences between different species is substantially independent.

9. The method of claim 1 wherein the n-mers to be tested contain sequence of size from 7 to 25 nucleotides long and wherein the set n-mers to be tested is generated randomly and contains from 10 to 1000,000 sequences.

10. The method or claim 1 wherein the set of n-mers to be tested is filtered or generated “quasi randomly” so all sequences have same or similar property selected from the group of properties consisting of: GC content, melting temperature (binding energy), presence or absence of same or similar pattern in certain position(s); inability to hybridize to themselves or other sequences in the set); presence of particular nucleotide or combination of nucleotides).

11. The method of claim 1 wherein the set of n-mers to be tested is generated “quasi randomly” so all sequences do not have particular pattern(s) (for example no sequences allow to have same nucleotide four or more times in lane).

12. The method in claim 1 wherein the set of n-mers is tested by using detection techniques comprising those selected from the group consisting of any DNA microarrays and parallel PCR, RT PCS, TaqMan, and other parallel detection techniques.

13. A nucleic acid hybridization-based biosensing device comprising a) a support having at least one surface and b) a collection of probe molecules attached to the surface, each probe being unique and comprising a plurality of oligonucleotide probe molecules, wherein the collection comprises a probe set.

14. A method of claim 1 for identifying viral, microbial and multi-cellular organisms, and of identifying individuals of the same species, based on the occurrences of short subsequences in the genomes.

15 The method of claim 1 in which randomly picked or quasi-randomly designed short oligomers are used in conjunction with parallel detection mechanisms selected from the group consisting of. DNA microarrays and parallel PCR and other parallel detection mechanisms, to form a device to conveniently identity the organisms in a biological sample.

16. The method of claim 1 used to identify viral, microbial and multi-cellular pathogens contained in a biological sample or to identify the presence or absence of any species, harmful or non-harmful, in any biological sample under other situations.

17. The method of claim 1 used to identify an individual among other individuals within the same species based on the differences in the occurrences of short subsequences in their genomes.

18. A method of claim 1 for identifying species or individuals within species comprising performing recognition analysis of present/absent patterns for selected n-mers, and comparing to such patterns for known moieties, to identity the biotechnical entity, without requiring prior knowledge of the genome sequences of the species or individuals to be identified.

19. A method of claim 18 comprising identifying an individual human being based on trace samples the human being leaves in a scene; and identifying/tracing individual livestock based on mcat sample in the food supply that may have been inflicted by certain diseases (e.g., mad cow disease).

20. All inventions described herein.
